(19) United States
(12) Patent Application Publication
(10) Pub. No.: US 2003/0125311 A1
(43) Pub. Date: Jul. 3, 2003

(54) Compounds capable of binding with the cytoskeleton

(76) Inventors: Etienne-Emile Baulieu, Neuilly sur Seine (FR); Paul Robel, Paris (FR); Esther Pellous, Paris (FR); Kokhi Murakami, Kanagawa (FR)

Correspondence Address: BURNS DOANE SWECKER & MATHIS L L P, POST OFFICE BOX 1404, ALEXANDRIA, VA, 22313-1404, US

(21) Appl. No.: 10/221,862

(22) PCT Filed: Mar. 19, 2001
(86) PCT No.: PCT/FR01/00816

(51) Int. Cl. A61K 31/56; C07J 53/00

(52) U.S. Cl. 514/169; 552/500

(57) ABSTRACT

The invention concerns the use of steroids and non-toxic compounds, capable of crossing the blood brain barrier characterised in that they are capable of binding the same site as pregnenolone on proteins constituting or associated with elements of the cytoskeleton and of displacing the MAP-bound pregnenolone, and of influencing the assembling and stabilisation of microtubules The invention is particularly useful for treating the nervous system, for fighting against ageing of cells containing MAPₛ, and for treating cancers.

The present invention relates to compounds capable of binding to proteins constituting or associated with elements of the cytoskeleton.

More particularly, it relates to their application for the manufacture of medicaments for the treatment of pathological states involving dysfunctions of these proteins.

The cytoskeleton is a complex network (see bibliographic reference (1) at the end of the specification) which includes the primary networks of three classes of cytoskeletal filaments (microtobules, microfilaments, and intermediary filaments) to which is added a microtrabecular network which comprises the majority of the proteins associated with the microtubules, microfilaments and intermediary filaments. The protein MAP₂ (family of Microtubule Associated Proteins described hereinafter) is a characteristic example of a component of this network: it has a region of binding to microtubules, but likewise to the microfilaments (2, 3) and to the intermediary filaments (4, 5).

The microtubules are hollow cylinders of 25 mm [sic] external diameter with a wall constituted by protofilaments, themselves formed by the stacking of globular α and β sub-units of tubulin (50 Kd). Associated in a periodic manner with the tubulin heterodimers are the proteins termed MAP, (microtubule associated proteins), the role of which is to facilitate the assembling of tubulin, but also the organization and the functions of the microtubular network.

Tubulin is very heterogeneous. Indeed, it comprises numerous isoforms arising both from the expression of several genes and from very numerous post-translational modifications (6).

The MAPₙ are divided into two groups, namely the “structural” MAPₙ involved in the assembly of the tubulins and the “motor” MAPₙ which use the microtubules as rails to effect the movement of certain molecules and organelles.

The structural MAPₙ which are an aim of the invention comprise several families of proteins, namely the A and B MAP₁ proteins (350 KD) coded by distinct genes, the A and B MAP₂ proteins of high molecular weight (300 Kd), the C ana D MAP₂ proteins of low molecular weight (70 Kd), the A and B MAP₃ proteins (180 Kd) present in neurons up to 10 days after birth, the ubiquitous A, B and C MAP₄ proteins (215 to 240 Kd), the Tau proteins (50-67 Kd or 110-120 Kd), and the STOP proteins (145 Kd), molecules which are very stabilizing for the microtubules.

In the neuron, the microtubules are involved in numerous functions (neural growth, maintenance of complex morphologies such as dendritic arborization, axonal transport of various molecules). For example, the MAP₁ proteins participate in dendritic arborization, while the tau proteins are essential for axonal growth. It is likewise known that the different isotypes of each MAP have specific localization and roles. MAP₂ B increases its expression from the fetal stage to the adult stage. MAP₂ A is expressed solely from a certain stage of development. MAP₂ C (2), which is expressed essentially in the early stages of development, is still present in the olfactory bulb (7) and in the photoreceptive cells of the retina (8) of the adult. MAP₂ D is localized in the region of the hippocampus from the fetal stage onward (3).

The microfilaments are polymers constituted by actin (43 Kd). They are very abundant in the cortical region of the axon, and are also found in the central region, where they participate in its dynamic architecture.

The intermediary filaments of the neurons are polymers constituted by a triplet of proteins (68, 150 and 200 Kd), and are present in differentiated neurons. Interconnected with the microfilaments and the microtubules, they help to maintain the neuronal architecture, the MAP₃ serving as a bridge between the three types of filamentous cytoskeletal structures.

After having revealed a high-affinity saturable binding with steroids in the cytosol of rat brain (fetus and young adult), the inventors have found that highly purified preparations of MAP₂ have the same binding characteristics.

Work in vitro by the inventors likewise demonstrated a specific action of steroids on the polymerization of the cytoskeletal structures.

It is at present considered that the effects of steroids are exerted via soluble intracellular receptors of another protein class. These receptors have been cloned, and are often classed as “nuclear” because of their mode of action as transcription factors at the level of the genome.

A series of observations published during the last 20 years indicates that the receptors for steroids likewise function at the level of the plasma membrane of the target cells, an important example of this being the action of neurosteroids at the level of the neurotransmitter receptors.

In revealing a direct interaction of steroids, more especially of pregnenolone (abbreviated PREG) with proteins constituting or associated with cytoskeletal elements, the inventors have revealed a new mechanism of action of these compounds, capable of being advantageously made use of for acting on all the functions involving the microtubules, intermediary filaments and microfilaments, including the growth, differentiation, and plasticity of neurons, the prevention and repair of neurodegenerative disorders and lesions, and more generally, all the phenomena of cell division.

In a general manner, this new mechanism of action concerns any non-toxic compound capable of passing through the blood-brain barrier, and capable of binding with the same site as PREG on the said proteins, or of displacing PREG, and of having an effect on the assembling and stabilization of microtubules.

The invention thus has as its object to provide compounds capable of binding to proteins constituting or associated with cytoskeletal elements such as identified by a screening method, comprising placing these compounds in contact with the said proteins under conditions permitting the establishment of a binding when the protein, for example MAP₂, is a receptor for the compound.

The invention is particularly aimed at the use of such compounds for the manufacture of medicaments for the treatment of pathological states involving these proteins.

The compounds which can be used according to the invention for the manufacture of medicaments are more especially steroid hormones (this term being used in the specification and the claims to cover their conjugates, such as sulfate esters or fatty acid esters), their precursors, their metabolites, free or conjugated, as well as their steroid or non-steroid analogs.

As a precursor of steroid hormones, there are mentioned in particular pregnenolone or its esters, particularly pregnenolone sulfate (PREGS), for which no intracellular receptor has been identified to date.

The analogs are pharmaceutical agents capable of increasing the formation of microtubules, of stabilizing them under the conditions given in the examples in relation to PREG, of entering into competition with PREG for binding to MAP₂, and of passing through the blood-brain barrier while remaining if possible sequestered in the nervous system (9, 10, 11).

They comprise polycyclic compounds, natural or enantiomeric, possibly in mixtures, their sulfates, or their lipid derivatives.

As novel products, the invention aims at the addition and/or substitution products formed from the said compounds, on the one hand, and proteins constituting or associated with cytoskeletal elements, on the other hand.

The invention particularly aims at the products of steroid—associated protein—microtubule couplings; in particular, steroid—MAP₂, if necessary in the form copolymerized with tubulin, or steroids—tau proteins in the form copolymerized with tubulin.

The invention particularly aims at the use of proteins constituting or associated with cytoskeletal elements as receptors of compounds such as those defined hereinabove.

The action of these compounds on the polymerization and stabilization of cytoskeletal structures, as illustrated in the examples, confers on them great advantages for the manufacture of medicaments.

The invention thus aims at the use of the said compounds for the manufacture of medicaments capable of specifically binding to proteins constituting or associated with cytoskeletal elements having a cytoplasmic or nuclear location, for the treatment of pathological states with dysfunctioning of these proteins.

It thus particularly aims at the use of the said compounds for the manufacture of medicaments for the treatment of the nervous system, central nervous system or peripheral nervous system. Such medicaments particularly permit stimulation of the differentiation/maturation of neurons.

The invention particularly aims at the manufacture of medicaments which can be administered in oral form in order to treat the central nervous system, or in injectable form for administration by the general route or in situ.

Other forms of administration fall within the scope of the invention, such as delay solutions or drug precursors conjugated with, for example, a fatty acid.

To increase biodisposability, it is likewise provided according to the invention to use the active principles in the form of appropriate derivatives, such as the esters of fatty acids.

In these applications, the medicaments manufactured according to the invention are advantageously used in association with growth factors (NGF) and/or cellular regulators such as interferon or the cytokines.

Applications of great interest comprise the prevention or the treatment of disorders such as Alzheimer's disease, dementias of vascular origin, the consequences of traumas and of vascular accidents at the level of the nervous system, and the neuro-degenerative diseases.

The invention likewise aims at the manufacture of medicaments by means of the said compounds, to combat the ageing of nerve cells or of any other cell type containing MAP₂, such as epithelial cells, thyroid cells, or endocrine glands, particularly those which form steroids.

In these applications for the treatment of the nervous system and of cell ageing, the said compounds can be used in association with compounds which have the effect of stabilizing the cytokeleton, such as Taxol® and Taxotére®.

To the extent to which the two types of molecules, steroid compounds, or steroid or non-steroid analogs, on the one hand, and Taxol® or Taxotére® or equivalent on the other hand, have a stabilizing effect on microtubules, it is possible to envisage a synergistic phenomenon if the two medicaments are used simultaneously. In this case, the doses of the two medicaments required for good effectiveness will be very much lower than those required when the medicaments are given as a mono-therapy.

According to another very advantageous aspect of the invention, the above-mentioned medicaments can be used for the treatment of cancers, in association with anti-cancer agents, and enable the effect of the latter to be modulated, in particular in cases of chemo-resistance.

Such associations with agents having a microtubular tropism are indeed able to potentiate the respective effects of the products.

In these different applications, the compounds used are associated with appropriate pharmacologically acceptable vehicles for the chosen form of administration. The unit doses are of the order of 100 to 500 mg, and the daily doses are of the order of 500 mg to 1 g for administration by general route, this dose being reduced for an in situ administration.

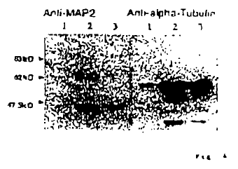

Other characteristics and advantages of the invention are given in the following description, with reference to FIGS. 1 to 7.

FIG. 1 relates to the equilibrium binding of PREG [³H] to fetal brain cytosol,

FIG. 2 relates to the chromatography of fetal brain cytosol by HPLC on a Mono Q ion exchange column,

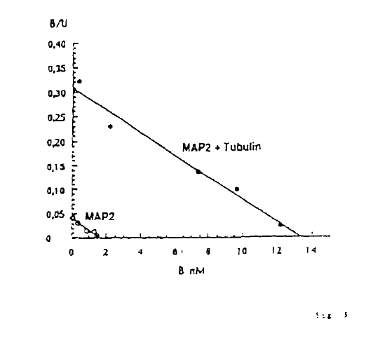

FIG. 3 relates to the chromatography of the Mono Q peaks by HPLC on a Superose 12 gel filtration column,

FIG. 4 relates to the immunoblot of cytosol and of fractions of one of the peaks eluted from the ion exchange and gel filtration columns,

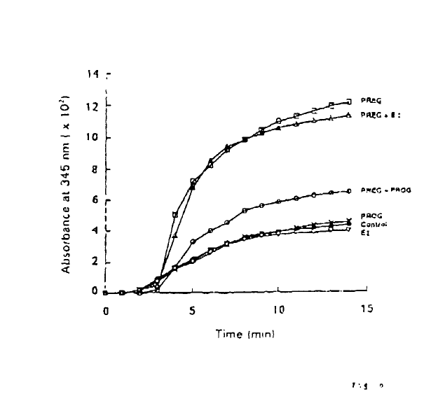

FIG. 5 relates to the equilibrium binding of PREG [³H] to purified MAP₂, complexed or not with purified tubulin.

FIG. 6 relates to the assembly kinetics of microtubules, and

FIG. 7 relates to the immunocytochemistry of fetal rat brain neurons.

### MATERIALS AND METHODS

Animals

Sprague-Dawley rats raised by Janvier (Le Genest St Isle, France) are used. The rats are under a 12-hour light/dark alternation (light at 8:00 a.m.) at 20° C. and allowed unlimited feed.

Pregnant females (the morning after copulation is indicated by E0) are kept in the animal house for at least 7 days before sacrifice at E18. Adult males 60 to 70 days old are also used.

The calf brains used are freed from meninges and large blood vessels. They are stored in a chilled isotonic saline solution.

The animals are treated in accordance with the provisions of the Directive of the Council of the European Union of Nov. 24, 1986 (86/609/EEC).

Materials

Steroids:

The PREG [7-³H] used (22.5 Ci/mole) used is a product sold by NEN (Boston, Mass., USA). Progesterone, pregnenolone, testosterone, corticosterone, triamcinolone acetonide, 17α-OH PREG, 1/α-OH progesterone and cholesterol are products of Sigma-Aldrich (St Ouentin Fallavier, France).

Enzymes:

Proteinase K (Merck, Darmstadt, Germany) and Pronase® are dissolved at 1 mg/ml in TNM buffer containing 10 mM of Tris, 0.1M of NaCl, 3 mM of MgCl₂ (pH 7.4). Deoxyribonuclease I and ribonuclease A are products of Sigma-Aldrich and are respectively adjusted to 100 IU/ml and 5 IU/ml. Phospholipase A2 (Roche Molecular Biochemicals) is diluted to 20 IU/ml in TNM.

Antibodies

Monoclonal anti-α-tubulin antibody N-356 is a product of Amersham Pharmacia Biotech (Freiburg, Germany). Monoclonal antibody 152 is an antibody produced by one of the inventors and reacts specifically with a MAP2 of high molecular weight.

The different antibodies are diluted in PBS containing 3% of BSA.

Methods

Preparation of Cytosol

Fetal brains are collected, and are rinsed, after the meninges have been removed, with a homogenization buffer at 4° C. [10 mM of Tris-HCl, at pH 8.5, containing 1.5 mM of EDTA, 1 mM of dithiothreitol, 10% of glycerol, Complete®, (a mixture of protease inhibitors, Roche Molecular Biochemical); termed TEDG buffer].

Adult male rat brains are collected after perfusion with PBS containing 2 IU/ml of heparin. The brains are weighed and homogenized with 4 vol. of homogenization buffer in Potter glass/Teflon® homogenizers, and centrifuged at 800 g for 10 min.

The supernatant is then centrifuged at 100,000 g for 1 h at 4° C. The samples are used immediately for the trials, or are stored in liquid nitrogen until the moment of their use.

Protein concentrations are determined using the Bio-Rad kit (Bio-Rad, Hercules, Canada) with BSA as reference standard.

Equilibrium Binding Constants

Just before the binding trial, each sample is purified with a dextran-carbon suspension, and 2 ml samples of supernatant (purified cytosol) are added to a series of tubes containing 1 to 500 nM PREG [³H], with or without unlabeled steroid in 1,000-fold excess.

The tubes are incubated at 45° C. for 30 min. The separation of bound and free steroid was carried out on a Sephadex LH-20® (Amersham Pharmacia Biotech) mini-column.

The counting of the radioactivity in the flasks was performed in a liquid scintillation spectrometer with attenuation correction (Tri-Carb 2100 TR®, Packard).

Nature of the Component Binding the Pregnenolone

1 ml of brain cytosol is incubated with 0.2 ml of an enzyme solution at 37° C. for 30 min. The binding activity is studied of samples (0.2 ml) which had undergone the enzyme treatment or control samples diluted with buffer only.

Competition Trials

In trials of competition between pregnenolone and different steroids for the binding studied, 0.2 ml of cytosol is incubated with 100 nM of PREG [³H] and a 1-, 10-, 100-, and 1,000-fold excess of non-radioactive steroid.

Anion Exchange Chromatography

The HPLC system (Amersham Pharmacia Biotech) was equipped with an anion exchange column (5 ml Econo-Pac High Q, Bio-Rad).

The column was equilibrated with a TEDG buffer containing 0.03% of CHAPS and 100 nM of PREG (TEDGCP buffer) at 4° C.

The cytosol (20 ml, 60 mg of protein) is applied and eluted at a rate of 1 ml/min with the TEDGCP buffer for 20 min.

Fractions of 2 ml are collected. A linear gradient of NaCl in TEDGCP is then applied to reach a NaCl concentration of 1M after 30 min., followed by the isocratic application of 1M NaCl in a TEDGCP buffer.

The appropriate fractions are reunited; the salts are eliminated by passage through Hi Trap® desalting mini-columns (Amersham Pharmacia Biotech).

Gel Filtration

The Superose 12® column (1×30 cm, Amersham Pharmacia Biotech) is equilibrated with TEDGCP buffer containing 0.15 M NaCl, at 4° C.

The throughput is adjusted to 0.25 ml/min. and 0.4 ml fractions are collected.

Monitoring the Purification of the Proteins

At each step of purification, the collected frictions are incubated with 100 nM PREG [³H] in the absence, or with a 100-fold excess, of non-radioactive PREG. The saturable binding is then determined.

Western Blots

Crude cytosol and partially purified samples (10 μg of protein) are separated by 10% SDS-PAGE, then transferred to nitrocellulose membranes. The membranes are blocked by incubation in 5% of skimmed milk containing 0.1% of Tween 20®, added to a Tris® buffer (Tris 10 mM, NaCl 50 mM, EDTA 2 mM, pH 8.8) for 1 h, at ambient temperature. All the washings are performed in a Tris buffer containing 0.1% of Tween 20® (TNT buffer). The membranes are then incubated with anti-tubulin antibodies (1:2,000) or anti-MAP2 monoclonal antibodies (1:1,000), at 4° C., for about 14 n.

Incubation with a mouse anti-IgG secondary antibody [fragment F(ab′)₂], coupled to peroxidase, 1/10,000, (Pierce, Rockford, USA) is performed in a TNT buffer containing 5% of milk, for 45 min., at ambient temperature. The signal is detected by an ECL chemiluminescence system (Amersham Pharmacia Biotech), with a Kodak Y-Omat film, following the manufacturer's instructions.

Preparation of Microtubule Proteins

Calf brains are washed twice in a L buffer (MES 0.1 M, EGTA 1 mM, EDTA 0.1 mM, MgCl 1 mM, dithiothreitol 1 mM, and PMSF 1 mM, pH 6.4).

The microtubules are prepared starting from calf brain or adult or fetal rat brain according to an assembly/disassembly method, as a function of the temperature, in vitro (12).

The calf tubulin is purified according to the protocol of Weingarten et al. (13), but the step using phosphocellulose is replaced by a cation exchange column chromatography (Fractogel, Merck) (14). The microtubules and the tubulin are prepared in L buffer, supplemented with 1 mM of GTP (buffer A).

The tau and MAP₂ proteins are purified according to the protocol of Fellous et al. (12) by heat denaturation of the microtubules and gel filtration on a column, Sephacryl 300® being used instead of the Ultrogel ACA 34. The tau proteins are then Further purified according to the method of Lindwall and Cole (15).

Measurement of the Assembling of Microtubules

This assembling is measured by monitoring the increases of turbidity with a UVICON® spectrophotometer (Kontron Instruments, Montigny-le-Bretonneux, France)

Culture of Neurons

Primary cultures of E17 fetal neurons are performed according to El-Etr et al. (16). Briefly, this method consists of spreading the dissociated cells (200,000 cells/cm²) on glass plates covered with poly-1-ornithine and cultured in a completely defined medium in 5% CO₂ at 37° C. The culture conditions used permit a practically pure neuron population to be obtained.

24 h after seeding, 1 micromolar pregnenolone (final ethanol concentration 0.01%), or the solvent alone, is added and cultures are continued for 2 days. The cells are fixed in 4% paraformaldehyde, then frozen at −80° C. The cells are duly rehydrated by passages of 5 min in ethanol at concentrations of 100, 90 and 70%. After 2 washings in PBS, for 5 min, the cells are incubated in PBS containing 3% of BSA and 0.1% of Triton X100®, at ambient temperature for 1 h.

After 4 washings in PBS, the cells are incubated at 37° C. for 1 h, then at 4° C. for about 14 h, in the presence of either anti-α-tubulin monoclonal antibody diluted 1/2,000, or anti-MAP2 monoclonal antibody diluted 1/1,000, or anti-double-cortin antiserum diluted 1/300.

After 3 washings in PBS, the secondary antibody is added at ambient temperature, for 45 minutes. This antibody is either mouse anti-IgG to rabbit IgG [fragment F(ab′)₂] conjugated to biotin, diluted 1/350 (Roche Molecular Biochemicals) or biotinized anti-rabbit IgG, diluted 1/300 (Boehringer).

After 3 washings, the peroxidase-streptavidine amplification complex (Dako, Denmark) is added for 30 min at ambient temperature.

The cells are washed and the AEC substrate (aminoethyl carbazole, Sigma-Aldrich, Saint-Quentin Fallavier, France) is deposited on the cells for 15 min. The reaction is stopped by the addition of distilled water. The cells are counterstained with hematoxylin and mounted on Glycergel®.

Electron Microscopy

After the polymerization of the microtubules induced by MAP₂ in the absence or presence of the substance to be tested, an aliquot portion of the experimental sample is deposited on a hydrophilic grid (300 mesh) covered with carbon. The grids are treated with an aqueous solution of 1% uranyl acetate. Observations are performed with a Phllips CM12 electron microscope.

Statistical Analyses

The equilibrium binding data are analyzed with the Mac Ligand program, adapted from Munson and Rodbard (17). The curves are traced with the kaleidograph™ 3.0 software (Abelbeck software, Ritme Informatique, Paris).

Results

Binding Sites of Pregnenolone in Rat Brain

The samples of cytosol (0.2 ml, 2.5 mg of protein/ml), previously desteroidized by adsorption on carbon/dextran, are incubated with increasing concentrations of PREG [³H] in a TEDG buffer at pH 8.5, at 40° C. for 30 min.

The bound steroid is separated by gel filtration on Sephadex LH20® at 0-4° C. The binding constants are determined with the Mac Ligand program.

FIG. 1 shows a Scatchard diagram of the specific binding.

A constant Kd of 31.0±0.8 nM is observed, and a maximum concentration of binding sites of 1.2±0.1 pmol/mg of protein (mean±standard error, n=3).

To study the effects of hydrolytic enzymes on the binding of PREG [³H], samples of 1 ml of cytosol incubated with 0.2 ml of an enzyme solution at 37° C. for 30 min were used. The results obtained, expressed as % of the control incubation (cytosol diluted in a buffer) are reported in Table 1 (mean±standard error, n=3).

TABLE 1

| Enzyme | Concentration/ml | | % of Control |
|---|---|---|---|
| Proteinase K | 0.17 | mg | 42.4 ± 7.5 |
| Pronase | 0.17 | mg | 40.5 ± 5.7 |
| Deoxyribonuclease 1 | 17 | IU | 99.4 ± 12.7 |
| Ribonuclease A | 0.8 | IU | 82.4 ± 9.2 |
| Phospholipase A2 | 33 | IU | 85.0 ± 7.3 |

It can be seen that the binding sites of the PREG [³H] are destroyed to the extent of 60% by proteinase K and Pronase®. On the contrary, no significant effect is observed with DNAase, RNAase and phospholipase A2.

To study the specificity of the binding of PREG [³H], different steroids were used.

The relative competition index was determined as ratios of the concentrations of the competitor (in the denominator) versus PREG causing a reduction of 50% of the specific binding of PREG [³H]×100 (mean±standard error, n=3). The results are given in Table 2.

TABLE 2

| Steroids | R.C.R. 50 |
|---|---|
| Pregnenolone | 100 |
| Preqnenolone sulfate | 78.8 ± 7.0 |
| Progesterone | 72.1 ± 17.7 |
| Δ5-pregnene-3β.20α-diol | 68.4 ± 15.3 |
| 3β-hydroxy-5α-pregnane 20-one | 18.4 ± 4.0 |
| Testosterone | 5.6 ± 2.8 |
| Dehydroepiandrosterone | 4.3 ± 2.6 |
| Estrone | 4.4 |
| Corticosterone | 2.8 |
| Androstenediol | 1.7 |
| Estradiol | 1.3 |
| Triamcinolone acetonide | 1 |
| 17α-hydroxy-pregnenolone | 0.85 |
| 17α-hydroxy-progesterone | 0.79 |
| DHEA sulfate | 0.045 |
| Cholesterol | 0.02 |

It can be seen that the best competitors for the binding of PREG [³H] are pregnenolone sulfate, progesterone, and Δ5-pregnene-3β, 20α-diol (20α-dihydro-pregnenolone).

Another steroid possessing a structure to that of PREG, namely 3β-hydroxy-5α-pregnane-20-one (5α-dihydro-PREG) is likewise shown to be an effective competitor, even though 5 times less active than PREG.

Among the other steroids tested, DHEA is a weak competitor, and DHEA sulfate has a practically negligible effect.

The preferred structures are C-21 steroids with a ketone group or an alcohol group in positions 3 and 20 and an unsaturated ring A or B conferring a flatter structure to the steroid skeleton.

Other trials performed with adult rat brains gave results of the same order, showing that the brain contains a compound which binds PREG, with binding constants similar to those of the fetal brain (Kd=50.7 nM, Bmax=0.75 pmol/mg of protein).

Purification of the Binding Protein of the Cytosol Chromatography on Mono Q® Column

The binding sites of pregnenolone are eluted by means of a Mono Q® ion exchange column. 60 mg of cytosol proteins in a TEDGCP buffer are loaded onto the column and eluted with a NaCl gradient. 2 ml fractions are collected. FIG. 2 reports the saturable binding of PREG [³H] PREG (cpm×10-4) (-∘-) and the concentration of proteins (mg/ml) (---⋄---) in each fraction.

The fractions of peak A (fractions 17-19: 4.5 mg of protein) and those of peak B (fractions 23-25: 1.1 mg of protein) are separately reunited. The salts of the 2 fractions are eliminated and the fractions are concentrated and subjected to gel filtration.

Chromatography on Superpose 12®

The peaks A and B are filtered on a Superose 12® column (elation with TEDGCP buffer containing 0.15 M of NaCl).

Fractions of 0.4 ml are collected. The specific binding of PREG [²H] is measured on each fraction of the column calibrated with nuclear weight labels. The results obtained are reported in FIG. 3, which gives for each fraction the saturable binding of PREG [³H] (-∘-) in cpm×10⁻⁴ and the concentration of proteins in mg/ml (---Δ---). The presence is noted of 2 peaks of 40-60 kDA (fraction A) and of more than 440 kDa (fraction B).

The presence of a component of very high molecular weight suggests that the binding protein of the cytosol could be a protein of very high molecular weight associated with the microtubules, which are the major constituents of the neuronal cytoplasm.

It was appropriate to verify the presence in the fraction A of components of the microtubule system. For this purpose, the peaks of PREG binding were subjected to a SDS-PAGE, followed by an immunoblot. The immunoblot of the cytosol and of the fractions of peak A eluted from the ion exchange columns and again passed through a gel filtration column is shown in FIG. 4. Track 1 corresponds to the crude cytosol; track 2 to the Mono Q® fraction A; track 3 to fraction A subjected to a new chromatography on Superose 12®. 10 μg of protein are deposited on tracks 1 and 2. The quantity of protein in track 3 is lower than the detection threshold.

It can be seen that the proteins which react immunologically with the anti-MAP2 and anti-tubulin (α and β) antibodies have been enriched in these partially purified preparations, and in fact in proportions compatible with the increase of activity of specific binding. The low molecular weight of the polypeptides recognized by the anti-MAP antibodies suggests that breakage of the 300 Kd (or 70 Kd) MAP protein(s) is produced during the purification of the cytosol protein.

Binding of PREG [³H] to Purified Microtubules of Rat Brain

The binding of PREG [³H] to microtubules prepared by polymerization/depolymerization cycles was determined.

The fetal microtubules have a constant Kd for PREG [³H] of 42.9 nM and a Bmax of 3.7 pmol/mg of protein, which represents a 3-fold enrichment with respect to the fetal brain cytosol.

The specificity of the ligand appeared to be unchanged (RCR: PREG(100)>PREGS (78.8)>PROG (72.1) >>testosterone (5.6)>DHEA (4.3)>estradiol (1.3)].

The adult microtubules have a constant Kd for PREG [³H] of 54.8 nM and a Bmax of 3.4 pmol/mg of protein, which represents a 4.5-fold enrichment compared to the adult brain cytosol.

Binding Activities of Pregnenolone to Purified MAP₂, Tubulin and Tau Protein

Purified calf brain tubulin (90 μg/ml), MAP₂ (45 μg/ml) or tau protein (25 μg/ml) is incubated with 100 nM of PREG [³H], with or without a 1,000-fold excess of non-radioactive PREG.

It is observed that neither tubulin, nor tau protein, nor rabbit IgG binds PREG [³H] in a saturable manner.

On the contrary, the MAP₂ fraction shows specific binding sites which increase when a co-incubation with tubulin is performed.

The equilibrium binding constants of purified calf MAP₂ are as follows: Kd=39.2 nM, Bmax=16 pmol/mg of MAP₂, while those of MAP₂-tubulin copolymers are: Kd=44.0 nM, Bmax=135 pmol/mg of MAP₂.

It thus appears that the binding of MAP₂ to tubulin does not modify the affinity for PREG, but on the contrary increases the concentration of binding sites by more than 8-fold.

The B/U values are reported in FIG. 5 as a function of B in nM, (-∘-) representing equilibrium binding of purified calf brain MAP₂, and (-∘-) the equilibrium binding of purified calf brain MAP₂ associated with purified calf brain tubulin (for the experimental details, see FIG. 1).

The molar concentration of binding sites is 14 nM, and that of MAP₂, based on a mean MW of 200,000, is about 200 nM.

It is thus observed that less than 10% of the molecules of MAP₂ can bind to PREG [³H].

The replacement of the buffer A by the TEDG buffer has no effect on the equilibrium binding constants of pregnenolone.

The specificity of binding of steroids to MAP₂-tubulin copolymers is very close to that of fetal microtubules [RCR: PREG (100)>PREGS (91.5)>PROG (81.3)>>estradiol (3.2)>testosterone and DMEA (2.3)].

Effects of Steroids on the Kinetics of Assembling of Microtubules

The microtubules are reconstituted in vitro with purified tubulin (1 mg/ml) and MAP2 (0.05 mg/ml) in buffer A.

The increase of absorbance is measured at 345 nm, at 37° C. and recorded every 30 sec. for 15 min.

The results are given in FIG. 6.

The assembling of microtubules is measured either in the absence of steroids (-⋄-); or in the presence of 500 nM of steroid: PREG (- -), progesterone (-X-), estradiol (-V-); or in the presence, at the same time, of PREG and progesterone (-∘-) or of PREG and estradiol (-Δ-) each at a concentration of 500 nM.

The increase of absorbance at 345 nm shows that PREG induces a strong increase of both the speed and the extent of the assembling of the tubulin. Estradiol, which does not bind to MAP2, is inactive. However, progesterone, which shows affinity for MAP2 of the same order as that of PREG, is likewise without effect. Contrary to estradiol, it counteracts the stimulative effect of PREG on the assembly of microtubules. PREG sulfate has effects similar to those of progesterone.

Analysis by Electron Microscope

Analysis was performed of microtubules polymerized in the presence of PREG to verify their appearance.

In the presence of calf brain MAP₂, calf brain tubulin is assembled into microtubules of normal appearance, that is, characterized by a remarkably constant conformation and a uniform diameter. The structure of the microtubules remains completely normal when MAP₂ induces the polymerization of tubulin in the presence of 500 nM of PREG.

Effects of Pregnenolone of Neuronal Cultures

Fetal rat brain neurons were studied by immunocytochemistry after 3 days of culture. The results are given in FIG. 7, where A, C, E correspond to a control culture of 3 days, B, D, F correspond to the addition of 1 μM of pregnenolone for the 2 last days of culture. In A and B, an anti-tubulin monoclonal antibody is added; in C and D, an anti-double-cortin polyclonal antibody, and in E and F, an anti-MAP₂ monoclonal antibody.

The staining with the monoclonal anti-α tubulin or anti-double-cortin antibodies does not show any difference between the control cultures and the cultures exposed to 1 μM pregnenolone for 2 days. On the contrary, the exposure to pregnenolone increases the immunolabeling of MAP₂ of the cell bodies and induces its extension to close nerve cells (see the arrows).

Study of the Competitive Capacity of Various Compounds with Pregnenolone

The results obtained with 11 steroid derivatives are reported in the following Table.

Pregnenolone (100%)

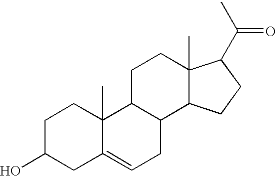

| | | Index of Competition | | |
|---|---|---|---|---|
| | Compounds with great | Cytosol | MT | MAP-2c + |
| | competitive capacity | (Rat) | (Beef) | Tubuline |
| 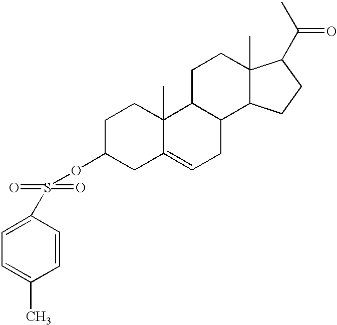 | PREG Tosylate | 107 | 121 | 129 |
| 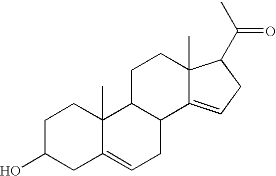 | 3β-OH-5, 14 Preqnadiene-20-one | 102 | 95 | |
| 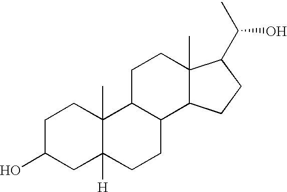 | 5a-Pregnama 3β, 20a-diol | 100 | 96 | |
| 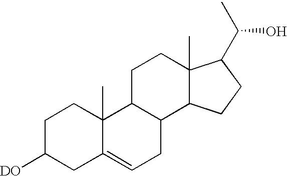 | Pregna-5-ene-3β, 20a-diol | 87 | 100 (beef) | |
| 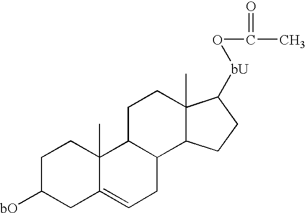 | 3β-OH-Pregna-5-ene-20-one-21- acetoxy (Sigma) | 80 (piglet) | 107 | |
| 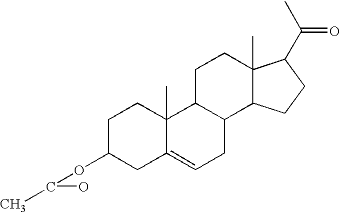 | PREG-acetate | 79 | | |
| 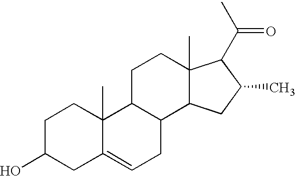 | PREG-16a-methyl | 80 | 88 | |
| 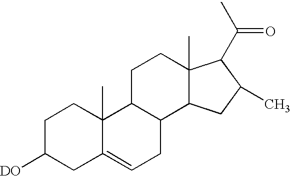 | PREG-16β-methyl | 63 | 83 (beef) | 97 |
| 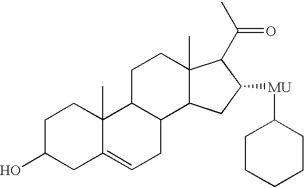 | PREG-1 Ga-cyclohexylamine | 59 | 81 (piglet) | |
| 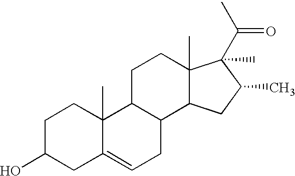 | Pregna-16a-17a-methylene | 62 | 59 | |
| 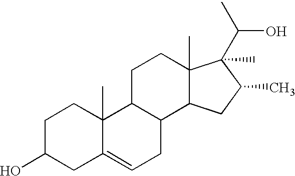 | Pregna-5-ene-3β,20β-diol- 16α,17a-methylene | 54 | | |

The results show that the steroids tested bind to the same site as PREG on the proteins constituting or associated with the cytoskeletal elements.

### BIBLIOGRAPHIC REFERENCES

1—WOLOSEWICK, J. J. and PORTER, K. R. (1979), J. Cell. Biol. 82, 114-139.

2—GRIFFITH, L. H. and POLLARD, T. (1982), J. Biol. Chem. 257, 9143-9151.

3—CORREAS, L. et al. J. (1990), Biochem. J, 269, 61-64.

4—AAMODT, E. J. and WILLIAMS, J. R., R. C. (1984), Biochemistry, 23, 6023-6031.

5—HIROKAWA, N. (1982)/J. Cell. Biol., 94, 129-142.

6—LUDUENA, R. F. (1998), Intern. Revi. Cytolo., 178, 207-275 .

7—VIERICK, C. et al. (1989), J. Neurosci, 9, 3547-3557 .

8—TUCKER, R. P. et al. (1988), Dev. Biol., 130, 423-434.

9—BODOR, N. et al. (1983), .J. Med. Chem., 26, 313-318.

10—BODOR, N. et al. (1984), J. of Pharm. Sciences, vol. 73, no. 3.

11—PARTDRIDGE, W. M. et al., 1979, J. Clin. Invest., vol. 64, 145-154.

12—FELLOUS A. et al., (1977), Eur. J. Biochem. 78, 167-174.

13. WEINGARTEN M. D. et al./Proc. Natl. Acad. Sci. USA (1975) 72, 1848-1852.

14—FROMES Y. et al., J. Protein Chem. (1996) 15, 377-388.

15—LINDWALL G. et al. (1984), J. Biol. Chem. 259, 12241-12245.

16—El Etr M. et al. (1989), J. Neurosci. 9, 1473-1480.

17—Munson P J and Rodbard D (1980), Anal. Biochem. 107, 200-239.

1. Non-toxic compounds capable of passing through the blood-brain barrier, characterized in that they are capable of binding to the same site as pregnenolone on proteins constituting or associated with cytoskeletal elements and of displacing pregnenolone bound to MAP₂, as well as having an effect on the assembly and the stablization of microtubules.

2. Addition products produced from compounds according to claim 1 or from steroids, on the one hand, and from proteins constituting or associated with cytoskeletal elements, on the other hand.

3. Addition products according to claim 2, characterized in that there are concerned steroids—associated proteins—microtubules, particularly steroids—MAP₂, if necessary in a form copolymerized with tubulin, or of steroids—tau proteins in a form copolymerized with tubulin.

4. Use of proteins constituting or associated with cytoskeletal elements as receptors of steroids or of compounds according to claim 1.

5. Use of steroids or of compounds according to claim 1, for the manufacture of medicaments for the treatment of pathological states involving proteins constituting or associated with cytoskeletal elements.

6. Use according to claim 3, characterized in that the steroid is pregnenolone or its sulfate.

7. Use according to claim 5 or 6 for the manufacture of medicaments for the treatment of the nervous system, central nervous system or peripheral nervous system.

8. Use according to claim 7, characterized in that the medicaments are presented in oral form for the treatment of the nervous system, or in injectable form, for administration by the general route or in situ, or else in the form of delay solutions or of drug precursors with conjugation, for example, to a fatty acid.

9. Use according to claim 7 or 8 for the manufacture of medicaments for the prevention or the treatment of disorders such as Alzheimer's disease, dementias of vascular origin, the consequences of traumas or vascular accidents at the level of the nervous system and neuro-degenerative diseases.

10. Use according to claim 5 or 6, for the manufacture of medicaments to combat the ageing of nerve cells, or of any other cell type containing MAP₂, such as epithelial cells, thyroid cells, or endocrine glands.

11. Use according to any one of claims 7-10 in association with compounds having a stabilizing effect on the cytoskeleton, such as Taxol®.

12. Use according to claim 5 or 6 for the manufacture of medicaments for the treatment of cancer, in association with anti-cancer agents.
